(19) United States
(12) Patent Application Publication
(10) Pub. No.: US 2023/0099725 A1
(43) Pub. Date: Mar. 30, 2023

(54) PROSTATE INNER LUMEN EXPANSION

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MN (US); Thomas J. Holman, Princeton, MN (US)

(21) Appl. No.: 17/935,470

(22) Filed: Sep. 26, 2022

Related U.S. Application Data
(60) Provisional application No. 63/261,715, filed on Sep. 27, 2021.

(51) Int. Cl. A61M 27/00; A61M 29/00

(52) U.S. Cl. CPC A61M 27/002; A61M 29/00; A61M 2210/166; A61M 2205/04; A61M 2205/0272

(57) ABSTRACT

Various embodiments disclosed relate to a device for insertion into the urethra for dilation. The present disclosure includes methods and devices including a device for at least partial insertion into a prostate, the device including first and second elongated members each having at least one magnetizable or magnetic element. The first and second elongated members are magnetizable to repel each other to dilate a lumen therebetween.

### PRIORITY CLAIM

This application claims the benefit of priority to U.S. Provisional Patent Application Ser. No. 63/261,715, filed Sep. 27, 2021, the contents of which are incorporated herein by reference in their entirety.

### BACKGROUND

Urethral stricture can involve narrowing of the urethra. Such a stricture can involve scarring and can restrict the flow of urine from the bladder and can cause various medical problems in the urinary tract. For instance, infection and inflation can occur with urethral stricture. Symptoms such as decreased urine stream, urinary tract infections, incomplete emptying of the bladder, among other symptoms, can manifest due to urethral stricture.

Often, urethral stricture can be caused by an enlarged prostate, often referred to as benign prostatic hyperplasia (BPH). BPH can occur with age in men. Where a urethral stricture is caused by an enlarged prostate, the enlarged prostate tissue can push inwards against the urethra to constrict the passage therein.

### SUMMARY OF THE DISCLOSURE

The present disclosure provides devices and methods for treating urethral stricture caused by an enlarged prostate. Such a device can include magnetic or magnetizable components that can be anchored in the prostate tissue and, when activated, allow for dilation of the urethra.

The discussed devices and methods can, for example, be used to address BPH, which is a common condition in older men. In BPH, an enlarged prostate gland can cause uncomfortable urinary symptoms, such as blocking the flow of urine out of the bladder. BPH can also cause other bladder, urinary tract, or kidney problems. Some approaches to treating BPH can include attempts to hold the urethra open through an otherwise constricted prostate.

Such conventional approaches can be invasive, and may not last a prolonged period of time, letting the constriction re-occur after the initial treatment. Moreover, many approaches are sudden instead of gradual. Other versions of luminal dilation can leave components in the body that are subject to encrustation and potential future infections.

For example, previous devices for urethra dilation used a tether for piercing the urethral wall, bound by plates. The tether pulled the plates towards each other. However, such a device would remain in the urethra, allowing encrustation issues. A similar previous device has used baskets which are temporarily inserted and expanded. However, these devices were a temporary solution that allowed the possibility of letting the urethra revert to the constricted state.

Thus, a minimally invasive approach for dilating a prostate-constricted urethra, as discussed below, is desired. Moreover, the disclosed approach can allow for permanent or controllable treatment, with a simple design. The discussed methods and devices can, for example, facilitate luminal expansion over time.

The devices and methods herein can include placing opposing magnetic or magnetizable elements in strategic positions in the prostate to hold open the prostatic urethra. The magnetic or magnetizable elements can be deployed in and around the prostate area surrounding the constricted urethral lumen, and aligned to repel on another. The magnetic repulsion can help widen the constricted urethra.

In an example, a device for at least partial insertion into a prostate can include a first elongated member sized and shaped for positioning within a first side of the prostate and a second elongated member sized and shaped for positioning within a second side of the prostate opposite the first side. Each of the first and second elongated members can include at least one magnetizable or magnetic element, such that the first and second elongated members are magnetizable to repel each other to dilate a lumen therebetween.

In an example, an apparatus for at least partial insertion into a prostate. The apparatus can include an insertion sheath comprising a lumen, a first elongated member for positioning on a first side of the prostate, and a second elongated member for positioning on a second side of the prostate opposite the first side. Each of the first and second elongated members can include at least one magnetizable or magnetic element, such that the first and second elongated members can be magnetized to repel each other to dilate a lumen therebetween. The lumen of the insertion sheath can be configured to hold the first and second elongated members during insertion of the apparatus into a patient anatomy.

In an example, a method of dilating a prostate can include inserting an applicator device comprising first and second elongated members into a lumen of the urethra, wherein the first and second elongated members each comprise at least one magnetizable or magnetic element, releasing the first and second elongated members into the prostate tissue on opposing sides of the lumen, and actuating the magnetizable or magnetic elements to induce repulsion between the first and second elongated members to dilate the lumen.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an enlarged prostate with a narrowed urethra in an example.

FIG. 2 illustrates a device for dilating a urethra in an example.

FIGS. 3A-3D illustrates a device with a delivery sheath for dilating a urethra in an example.

FIGS. 4A-4B illustrates magnetizable members for dilating a urethra in an example.

FIG. 5 illustrates a device for dilating a urethra in an example.

FIG. 6A-6D illustrates magnetizable members for dilating a urethra in an example.

FIG. 7 illustrates a method of inserting a device for dilating a urethra in an example.

### DETAILED DESCRIPTION

The present disclosure describes, among other things, a device and methods for treating a prostate-constricted urethra. The device is minimally invasive and helps dilate the urethra in a permanent or controllable fashion. The device can have a simple design and be made of ferromagnetic materials to induce a magnetic push to dilate the lumen.

For example, a device can include two or more magnetic or magnetizable elements. The magnetic or magnetizable elements can be positioned within a prostate with minimal or no exposure to the urine tract. In some cases, the magnetic or magnetizable elements can be permanent implants. In some cases, magnetic or magnetizable elements can be biodegradable and allow for tissue remolding. Magnetic flux lines from the magnetic or magnetizable elements can cause the magnetic or magnetizable elements to repel each other, thereby dilating a constricted urethra situated therebetween. Such magnetic or magnetizable elements can vary in design, shape, and size, as discussed below.

The use of magnetic or magnetizable elements to dilate a urethra lumen along the prostate can allow for a simple design, continued luminal expansion over time, and can help reduce risk of encrustation.

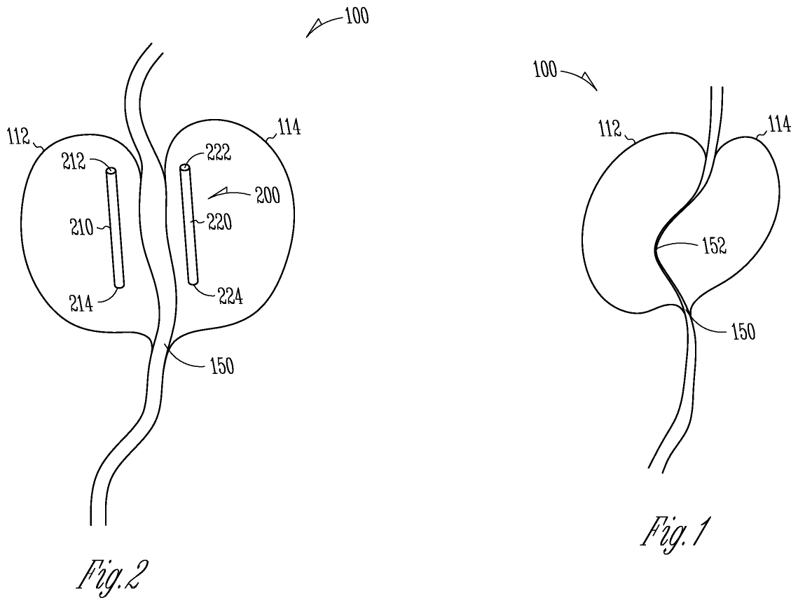

FIG. 1 illustrates an enlarged prostate 100 with a narrowed urethra 150 in an example. The prostate 100 can include portions 112, 114, surrounding the urethra 150 with restricted portion 152. The prostate 100 is a gland that helps produce semen. As men age, their prostate can grow in size. In some cases, a prostate that is too large can cause problems. An enlarged prostate can also be referred to as BPH.

The prostate 100 surrounds the urethra 150. When the prostate 100 is enlarged, the prostate tissue (e.g., portions 112 and 114) can push inwards towards the urethra 150 and cause a restriction of the lumen through which urine flows. This can result in a number of symptoms, including a frequent need to urinate, such as at night, trouble starting a urine stream, a weak urine stream, blood in the urine, and frequent urges to urinate, even if urination has just occurred. Such as restricted lumen can be addressed, for example, by the devices and methods discussed below.

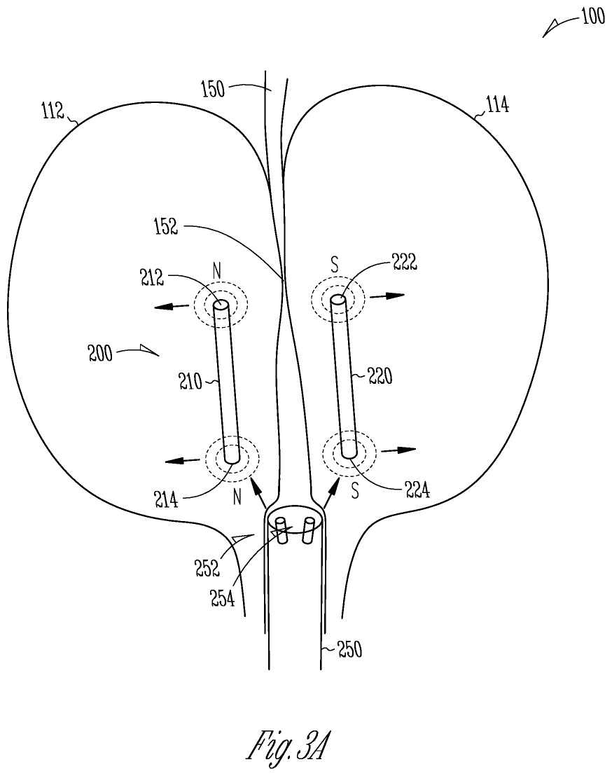

FIG. 2 illustrates a device 200 for dilating a urethra 150 in an example. The device 200 can include two or more magnetizable or magnetic elements. In the device 200 shown in FIG. 2, the elements can include a first rod 210 with ends 212, 214, and a second rod 220 with ends 222, 224.

Shown in FIG. 2, two rods 210, 220, can be situated in and secured in portions of the prostate 112, 114, opposite each other, on either side of the urethra 150. In some cases, an additional third rod can be used. The rods 210, 220, can help stretch open the urethra 150 to correct restriction. In some cases, the rods 210, 220, can hold the area open. The rods 210, 220 can be deployed either for permanent placement, or removed after a desired amount of time. In some cases, the rods 210, 220, can be biodegradable. In some cases, the magnetic or magnetizable components can be embedded in a biodegradable material, such that after degradation, the magnetic or magnetizable components remain in the prostate tissue.

Each of the rods 210, 220, can include one or more magnetic or magnetizable components. In some cases, the magnetic or magnetizable components can be permanent magnets. In some cases, the magnetic or magnetizable components can be controllable magnets. In the case of controllable magnets, the element can, for example, be turned on and off extracorporeally, such as with a mechanism outside the body near the anatomical area. For example, a magnetic wand can be used to turn the magnetic or magnetizable components on or off. In some cases, the magnetic or magnetizable components can be turned off and on using an internal transluminal mechanism, such as a rod in the urethra.

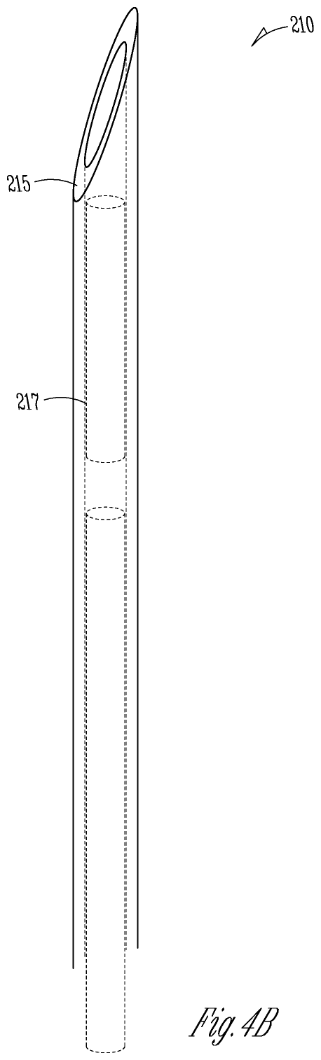

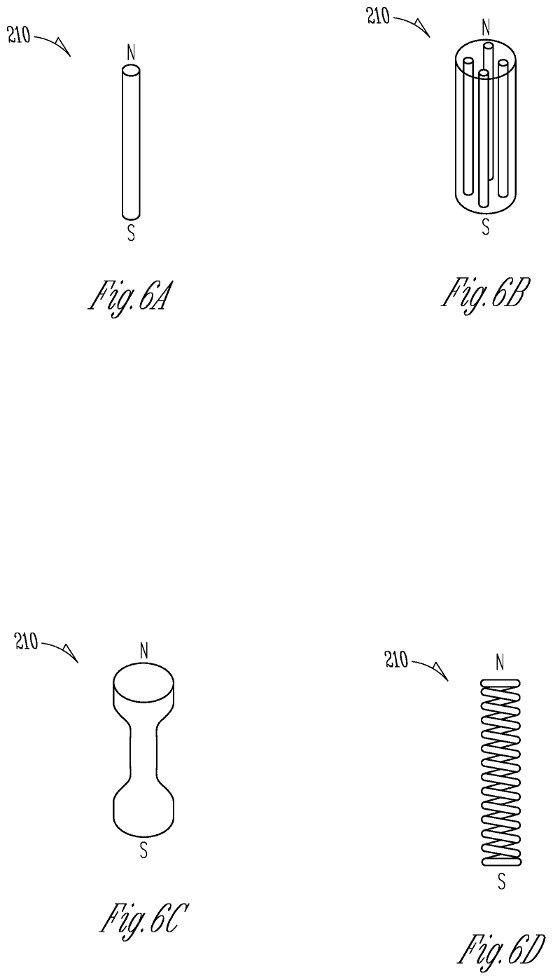

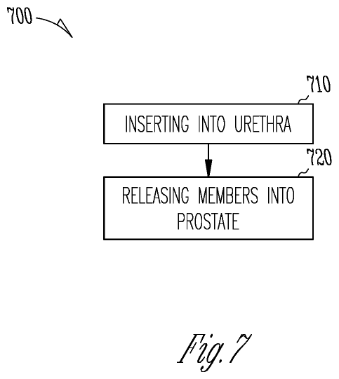

In an example, the rods 210, 220, can be iron containing rods. The rods 210, 220, can be implanted into the prostate on either side of the urethra lumen. A magnet actuator can then be passed transluminally through the urethra to magnetize the rods 210, 220, such as during initial deployment. In this way, the rods 210, 220, become magnetized and repel each other, stretching the urethra lumen open. In some cases, the rods 210, 220, can be magnetic members in other forms, such as discussed below in references to FIGS. 4A-4B and 6A-6D.

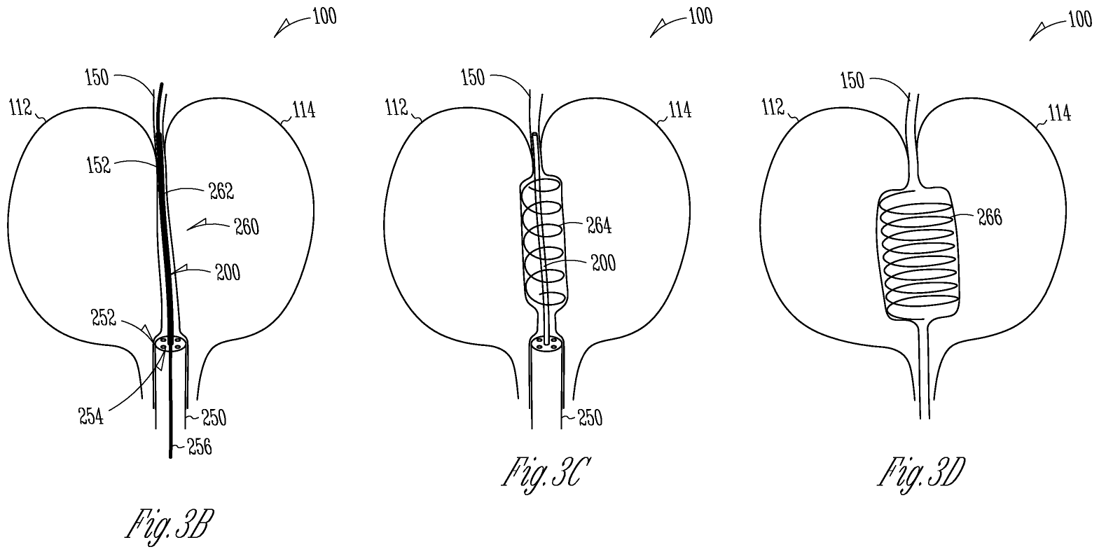

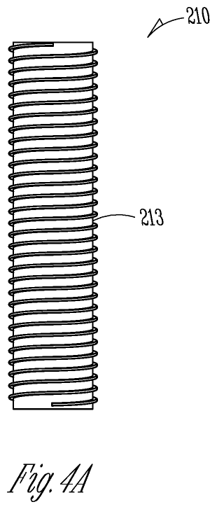

FIGS. 3A-3D illustrate a device 200 with a delivery sheath 250 for dilating a urethra 150 in an example. The device 200 can be similar to the device 200 discussed above with respect to FIGS. 1 and 2; FIGS. 3A-3D show how a sheath can be used with the device 200. Any of the BPH devices discussed above or below can be modified to include such a sheath. During application by a practitioner to a client's urethra, these magnetizable members 210, 220, can be transluminally implanted through the urethral wall. Various example delivery mechanisms are shown in FIGS. 3A-3D.

Shown here, the prostate 100 can include portions 112, 114, surrounding the urethra 150 with restricted portion 152. The device 200 can include two or more magnetizable or magnetic elements. In the device 200, the elements can include the first rod 210 with ends 212, 214, and the second rod 220 with ends 222, 224. The device 200 can be applied with the delivery sheath 250 having a distal end 252 with an opening 254 and a guidewire 256. The device 200 can be placed, for example, with a delivery system 260.

As shown in FIGS. 3A-3D, a guidewire 256 can be passed into the urethra 150, and a deployment device (e.g., delivery sheath 250) can be passed over the guidewire 256. The deployment device 250 may include a balloon mechanism that expands the urethral lumen to facilitate implantation of the rods or other magnetic members 210, 220. The ends 212, 214, and 222, 224 of the members 210, 220, can be anchored within the prostate tissue once deployed. In some examples, the ends 212, 214 can have opposing magnetic charges from the ends 222, 224, such that the first member 210 and the second member 220 repel each other when magnetized. Other deployment systems can be alternatively used.

For example, in FIG. 3A, the delivery sheath 250 can be an elongated shaft or tube having a proximal end and a distal end 252, such as including a lumen with the opening 254 at the distal end 252. The magnetizable device 200 with first rod 210 and second rod 220 can fit within or partially within the lumen of the delivery sheath 250, such that a practitioner can insert the delivery sheath 250 into a patient's urethra with the device 200 therein. In some cases, the delivery sheath 250 can be expandable to allow for insertion of the device 200 into the urethra.

The delivery sheath 250 can be sized and shaped for such insertion into a urethra, without exposing the device 200 to the urinary tract during insertion. In some cases, the delivery sheath 250 can be manipulated and placed by the guidewire 256. The sheath 250 can be manipulatable by the practitioner such that, after insertion to a desired location in the urethra (e.g., a location near the prostate gland subject to stricture), the practitioner can push the device 200 out of the delivery sheath into the urethra.

When the sheath 250 is situated far enough into the urethra to target the enlarged prostate 100 tissue, the practitioner can actuate the device 200 to insert the rods 210 and 220 into the urethra lumen, exiting out the opening 254 at the distal end 252 of the sheath 250. Once placed, the rods 210, 220, can be magnetized to induce separation between the two rods 210, 220. Magnetization can be done, for example, with an actuating piece, such as an internal or external magnet configured to change (or induce) the magnetic field and induce the rods 210, 220, to repel each other. The rods 210, 220, can then migrate away from each other within the prostate tissue to open the restricted portion of the urethra, where the rods 210, 220 can be anchored by their respective ends 212, 214, 222, 224. In some cases, the ends 212, 214, 222, 224 can include one or more lateral protrusion for inhibiting migration of the magnetizable members 210, 220. Such lateral protrusions can be configured to press against or engage one or more physiological structures. In some cases, such lateral protrusions can be located elsewhere along the elongated bodies of the magnetizable members 210, 220.

In some cases, the delivery sheath 250 can further include a delivery system 260. For example, in FIG. 3B, a delivery system 260 can include a thin delivery portion 262 that is non-fixable and helps to guide the sheath 250 with the device 200 along the guidewire 256. In another example, shown in FIGS. 3C-3D, the delivery system 260 can include an expandable delivery portion 264 or 266 configured to aid in opening the urethra for placement of the device 200. The delivery portion 264 or 266 can include, for example, a wire or other collapsible configuration that, when expanded, creates a spiral, conical, or cylindrical pattern configured to push the walls of the lumen of the urethra open. In some cases, such a delivery portion 264 or 266 can be integrated with the device 200 and can be magnetizable such that it can be embedded and hold the prostate tissue aside to prevent stricture of the urethra. In other cases, it can allow for room to place a separate device 200, such as the rods 210, 220, discussed above.

In some cases, the magnetizable elements (e.g., rods 210, 220) of the device 200 can be deployed instead with a balloon. Such a balloon could be inserted through the delivery sheath 250 with the device 200. Once inserted, the balloon could be pushed out into the urethra in the restricted portion, and inflated to push the walls of the urethra open for insertion of the device 200. The device 200 rods 210, 220, could then be placed in the urethra at the appropriate location. The balloon could be deflated, then removed. After removal of the balloon, the device 200 rods 210, 220, can be magnetized and pushed through the prostate 100 tissue to allow for dilation of the urethra 150 lumen. The delivery sheath or system 250 can be used to encourage the placement of the magnetizable members 210, 220.

FIGS. 4A-B illustrates devices for dilating a urethra 150 in an example with example magnetizable members 210. The magnetizable members 210 can be, for example, rods, as discussed above, or of other shapes, sizes, and iterations, suitable for dilating the urethra.

The magnetic or magnetizable members 210, 220 may be in various forms. In some cases, the members 210, 220, can include a plurality of permanently magnetized members. Alternatively, the implanted members 210, 220 may be magnetized, or demagnetized, at desired times. For example, ferrous members 210, 220 may be implanted, followed by a magnet transluminally passed through the urethra to magnetize the members individually or collectively. In some cases, an extracorporeal magnet can be used to activate or deactivate the magnetic repulsion. Such embodiments allow a physician and patient to turn the expanding mechanism on and off when needed or otherwise desired. In some cases, the magnetizable members 210, 220, can be drug coated, such as for anti-inflammatory purposes.

Shown in FIG. 4A, an example magnetizable member 210 can include a screw-like feature or threading 213 to enable rotational insertion of the member 210. Shown in FIG. 4B, an example magnetizable member 210 be situated in a needle-tipped or angled shaft 215 that can be pushed into the prostate tissue. In some cases, the magnetic member 210 can be pushed out of such a shaft 215 after insertion. In some cases, the magnetic member 210 can include a plurality of magnetizable elements 217 in the shaft 215 for insertion.

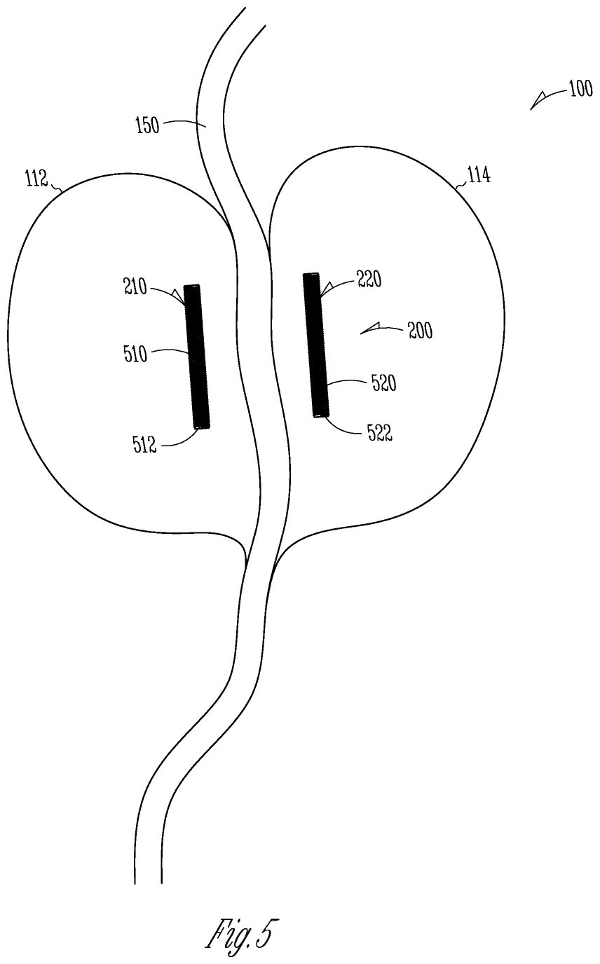

FIG. 5 illustrates a device 200 for dilating a urethra 150 in an example. Shown in FIG. 5 are example variations on the implant magnetizable members 210, 220. For example, the magnetizable members 210, 220 can have a coating or impregnation to release a drug over time, such as an anti-inflammatory or anti-proliferation drug. In some examples, the drug can be “refilled” in the magnetic member or in a retractable insert associated with the magnetic member.

In another example, instead of rods, the magnetizable members 210, 220, can be made of a biodegradable gel 510, 520 filled with magnetic or magnetizable particles 512, 522. In this case, after insertion, a large portion of the magnetizable members 210, 220 degrades, such as the biodegradable gel. The filler magnetic particles 512, 522 can be left in the implanted space to hold the prostate tissue apart and continue dilation of the urethra.

In some cases, the magnetizable members 210, 220, can be constructed with exterior surfaces to allow visualization thereof, such as by ultrasound. In some case, the magnetizable members 210, 220, can be made of materials that allow for external heating when exposed to particular outside forces, such as ultrasonic vibration, magnetic field oscillation, or other forces. In some cases, such forces can be used to migrate the magnetizable members 210, 220, further apart from each other to continue to dilate the urethra.

FIGS. 6A-6D illustrate example magnetizable members 210 for dilating a urethra 150 in an example. In some cases, the magnetic members 210 can be a simple smooth rod made of solid metal, shown in FIG. 6A. In some cases, the magnetic members can be made of a particulate within a polymer. In some cases, multiple rods can collectively be used at a location to allow more flexibility when magnetizing than a single rod, shown in FIG. 6B.

The cross section of the magnetizable members 210 can be geometrically uniform along its length, or can have a varying radial geometry along the cross section. For example, shown in FIG. 6C, a “dumbbell-shaped” magnetic member can have larger north and south heads to exert a larger force, with the middle section being narrower to facilitate flexibility and compliance. In some cases, the magnetizable members 210 can be of a spring-like or spiral pattern, shown in FIG. 6D.

In some cases, the magnetizable members can be of differing shapes, such as ribbon shapes, rods, spiral, braided, or coiled shapes. In some cases, the magnetizable members can be of approximately equal lengths to each other. In some cases, the magnetizable members can be of unequal lengths to each other. In some cases, the magnetizable members can include one or more arcuate sections. In some cases, the magnetizable members can each include two or more interwoven strands. In some cases, the magnetizable members can be of approximately uniform thickness. In some cases, the magnetizable members can be of varying thickness. In some cases, the magnetizable members can each include multiple segments.

FIG. 7 illustrates a method 700 of inserting a device for dilating a urethra in an example. The method 700 can include blocks 710 to 730.

First, at block 710, an applicator can be inserted into a urethra. The applicator device can include first and second elongated members. The first and second elongated members can each include at least one magnetizable or magnetic element.

Next, at block 720, the first and second elongated members can be released into the prostate tissue on opposing sides of the lumen. Subsequently, the first and second elongated members can be actuated the magnetizable or magnetic elements to induce repulsion between the first and second elongated members to dilate the lumen.

In some cases, the method 700 can include anchoring the first and second elongated members in the prostate tissue on opposing sides of the lumen. In some cases, the method 700 can include de-magnetizing the magnetizable or magnetic elements to reduce repulsion between the first and second elongated members.

### Various Notes & Examples

Example 1 can include a device for at least partial insertion into a prostate, the device comprising: a first elongated member sized and shaped for positioning within a first side of the prostate; and a second elongated member sized and shaped for positioning within a second side of the prostate opposite the first side, each of the first and second elongated members comprising at least one magnetizable or magnetic element, such that the first and second elongated members are magnetizable to repel each other to dilate a urethra therebetween.

Example 2 can include Example 1, wherein the at least one magnetizable or magnetic element includes one or more magnetizable or magnetic elongate members.

Example 3 can include Example 1, wherein the first and second elongate members comprise elongated ribbon shapes.

Example 4 can include Example 1, wherein the first and second elongate members comprise rods.

Example 5 can include Example 1, wherein the first and second elongate members comprise relatively equal lengths.

Example 6 can include Example 1, wherein one of the first and second elongate members is longer than the other.

Example 7 can include Example 1, wherein at least one of the first and second elongate members comprise one or more arcuate sections.

Example 8 can include Example 1, wherein at least one of the first and second elongate members comprises a lateral protrusion for inhibiting migration of the elongate members by pressing against or engaging a physiological structure.

Example 9 can include Example 1, wherein the first and second elongate members form braided or interwoven strands.

Example 10 can include Example 1, wherein at least one of the first and second elongate members includes a thickness varying along a length of at least a portion of the at least one of the magnetizable or magnetic elongate members.

Example 11 can include Example 1, wherein at least one of the first and second elongate members comprise multiple segments.

Example 12 can include Example 1, wherein at least one of the first and second elongate members comprises a spiral shape.

Example 13 can include an apparatus for at least partial insertion into a prostate, the apparatus comprising: an insertion sheath comprising a lumen; a first elongated member for positioning on a first side of the prostate; and a second elongated member for positioning on a second side of the prostate opposite the first side, each of the first and second elongated members comprising at least one magnetizable or magnetic element, such that the first and second elongated members can be magnetized to repel each other to dilate a lumen therebetween; wherein the lumen of the insertion sheath is configured to hold the first and second elongated members during insertion of the apparatus into a patient anatomy.

Example 14 can include Example 13, further comprising one or more inflatable portions configured to move the first and second elongated members into patient anatomy when the insertion sheath is inserted.

Example 15 can include Example 13, further comprising a magnetic applicator for magnetizing the first and second elongated members.

Example 16 can include Example 15, wherein the magnetic applicator is configured to be drawn through the insertion sheath to actuate the at least one magnetizable or magnetic elements to provide magnetic repulsion to encourage the first and second elongated members to repel each other.

Example 17 can include Example 15, wherein the magnetic applicator is configured to be applied externally to actuate the at least one magnetizable or magnetic elements provide magnetic repulsion to encourage the first and second elongated members to repel each other.

Example 18 can include a method of dilating a prostate, comprising: inserting an applicator device comprising first and second elongated members into a lumen of a urethra, wherein the first and second elongated members each comprise at least one magnetizable or magnetic element; releasing the first and second elongated members into tissue of the prostate on opposing sides of the lumen; and actuating the magnetizable or magnetic elements to induce repulsion between the first and second elongated members to dilate the lumen.

Example 19 can include Example 18, further comprising anchoring the first and second elongated members in the prostate tissue on opposing sides of the lumen.

Example 20 can include Example 18, further comprising de-magnetizing the magnetizable or magnetic elements to reduce repulsion between the first and second elongated members.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for at least partial insertion into a prostate, the device comprising:
a first elongated member sized and shaped for positioning within a first side of the prostate; and
a second elongated member sized and shaped for positioning within a second side of the prostate opposite the first side,
each of the first and second elongated members comprising at least one magnetizable or magnetic element, such that the first and second elongated members are magnetizable to repel each other to dilate a urethra therebetween.

2. The device of claim 1, wherein the at least one magnetizable or magnetic element includes one or more magnetizable or magnetic elongate members.

3. The device of claim 1, wherein the first and second elongate members comprise elongated ribbon shapes.

4. The device of claim 1, wherein the first and second elongate members comprise rods.

5. The device of claim 1, wherein the first and second elongate members comprise relatively equal lengths.

6. The device of claim 1, wherein one of the first and second elongate members is longer than the other.

7. The device of claim 1, wherein at least one of the first and second elongate members comprise one or more arcuate sections.

8. The device of claim 1, wherein at least one of the first and second elongate members comprises a lateral protrusion for inhibiting migration of the elongate members by pressing against or engaging a physiological structure.

9. The device of claim 1, wherein the first and second elongate members form braided or interwoven strands.

10. The device of claim 1, wherein at least one of the first and second elongate members includes a thickness varying along a length of at least a portion of the at least one of the magnetizable or magnetic elongate members.

11. The device of claim 1, wherein at least one of the first and second elongate members comprise multiple segments.

12. The device of claim 1, wherein at least one of the first and second elongate members comprises a spiral shape.

13. An apparatus for at least partial insertion into a prostate, the apparatus comprising:
an insertion sheath comprising a lumen;
a first elongated member for positioning on a first side of the prostate; and
a second elongated member for positioning on a second side of the prostate opposite the first side,
each of the first and second elongated members comprising at least one magnetizable or magnetic element, such that the first and second elongated members can be magnetized to repel each other to dilate a lumen therebetween;
wherein the lumen of the insertion sheath is configured to hold the first and second elongated members during insertion of the apparatus into a patient anatomy.

14. The apparatus of claim 13, further comprising one or more inflatable portions configured to move the first and second elongated members into patient anatomy when the insertion sheath is inserted.

15. The apparatus of claim 13, further comprising a magnetic applicator for magnetizing the first and second elongated members.

16. The apparatus of claim 15, wherein the magnetic applicator is configured to be drawn through the insertion sheath to actuate the at least one magnetizable or magnetic elements to provide magnetic repulsion to encourage the first and second elongated members to repel each other.

17. The apparatus of claim 15, wherein the magnetic applicator is configured to be applied externally to actuate the at least one magnetizable or magnetic elements provide magnetic repulsion to encourage the first and second elongated members to repel each other.

18. A method of dilating a prostate, comprising:
inserting an applicator device comprising first and second elongated members into a lumen of a urethra, wherein the first and second elongated members each comprise at least one magnetizable or magnetic element;
releasing the first and second elongated members into tissue of the prostate on opposing sides of the lumen; and
actuating the magnetizable or magnetic elements to induce repulsion between the first and second elongated members to dilate the lumen.

19. The method of claim 18, further comprising anchoring the first and second elongated members in the prostate tissue on opposing sides of the lumen.

20. The method of claim 18, further comprising de-magnetizing the magnetizable or magnetic elements to reduce repulsion between the first and second elongated members.
